(19) United States
(12) Patent Application Publication
(10) Pub. No.: US 2012/0039820 A1
(43) Pub. Date: Feb. 16, 2012

(54) ANTIBACTERIAL COMPOSITION COMPRISING 4-ISOPROPYL-3-METHYLPHENOL AND ZINC IONS

(75) Inventors: Alison Axe, Surrey (GB); David Bradshaw, Surrey (GB)

(73) Assignee: Glaxo Group Limited, Greenford, Middlesex (GB)

(21) Appl. No.: 13/262,268

(22) PCT Filed: Apr. 1, 2010
(86) PCT No.: PCT/EP10/54393

§ 371 Date: Sep. 30, 2011

(30) Foreign Application Priority Data
Apr. 3, 2009 (GB) 0905863.7

(51) Int. Cl. A61K 8/46; A01N 59/16; A01P 1/00; A61K 8/58; A61P 31/04; A61K 31/315; A01N 55/02; A61Q 11/00; A61K 33/30

(52) U.S. Cl. 424/52; 424/49; 424/641; 514/494

(57) ABSTRACT

A composition such as an oral care composition comprising an antibacterial system comprising 4-isopropyl-3-methyl phenol (IPMP), a source of zinc ions and an anionic surface active agent is described.

This invention relates to a composition comprising an antibacterial system comprising 4-isopropyl-3-methylphenol (IPMP), a source of zinc ions and an anionic surfactant. Suitable compositions include disinfecting compositions, pharmaceutical compositions, or personal care compositions for oral, throat and skin care. Of particular interest are oral care compositions comprising the antibacterial system which are of use in maintaining healthy gums and teeth, and are of use in combating (ie helping to prevent, inhibit and/or treat) oral health conditions caused or exacerbated by the presence of bacteria present in the oral cavity. Such conditions include periodontal (gum) diseases, dental caries (tooth decay), halitosis (oral malodour), dental plaque and dental calculus.

Several hundred species of bacteria, together with some fungal species, viruses and occasionally protozoa form the oral microflora, most obviously visible as the grainy off-white deposits found on tooth surfaces—which is known as dental plaque. Most of the time, the oral microflora exists in a healthy and stable relationship with the host, and may even provide a benefit by providing protection—termed colonisation resistance—against invasion of the oral cavity by potentially pathogenic micro-organisms which are constantly ingested. However, the oral microflora is also the aetiological agent of two of the commonest diseases affecting man—dental caries (tooth decay) and periodontal (gum) diseases.

Dental caries results from the repeated consumption of sugar in the diet, which is converted by a number of oral bacteria (especially members of the Streptococcus group of bacteria, and in particular Streptococcus mutans) residing on tooth surfaces to lactic acid which demineralises dental enamel.

Periodontal diseases, in contrast, result from accumulation of dental plaque at the gum margin, and are associated with an increase in proportions of some components of the microflora (especially anaerobic bacteria). This increased plaque mass provokes a host immune response, causing inflammation of the gum tissues, which may include bleeding. This is termed gingivitis. Gingivitis may lead to the formation of a gingival pocket, wherein more bacteria may accumulate in the pocket between the tooth and the inflamed gum. If left unchecked, this sub-gingival plaque may lead to the development of more serious gum disease—periodontitis—which ultimately may lead to tooth loss. Other by-products of the oral microflora may lead to bad breath—a common, but socially distressing condition. Bacterial plaque may become more firmly attached and calcified on dental surfaces, forming dental calculus. Dietary components such as coffee, tea and red wine can then cause this calculus to become stained in an unsightly way.

It follows from the above discussion that the complete elimination of the oral microflora is neither feasible nor desirable. Instead, strategies are aimed at regularly cleaning the oral cavity to reduce the quantities of dental plaque, or restricting the re-growth or development of the oral microflora, so that it remains in a state compatible with dental and gingival health.

Regular mechanical cleaning by toothbrushing is the key to reducing the quantity of dental plaque and thus maintaining gingival health. The use of chemical agents as an adjunct to this physico-mechanical control of plaque has been advocated for a number of years. Chemical plaque control enhances mechanical plaque control by direct killing of plaque bacteria, by inhibiting the regrowth of plaque, by reducing the metabolic activity of plaque or by a combination of all three mechanisms. In this way, plaque may be maintained at levels which are compatible with gingival health. In the absence of an increased gingival plaque challenge, the gum margin may remain tight, thus affording protection to the sub-gingival parts of the tooth and other tissues. In this way a whole range of potentially deleterious oral health effects can be avoided.

Accordingly it has become highly desirable to include within an oral healthcare product materials that will kill, inhibit or retard the growth or metabolism of bacteria found in the oral cavity.

Antibacterial agents are often found in oral healthcare products. Commonly included are the cationic compounds chlorhexidine, benzalkonium chloride and cetyl pyridinium chloride. Nonionic compounds include halogenated diphenyl ether compounds such as Triclosan, halogenated carbanilides such as trichlorocarbanilide, and phenolic compounds such as thymol, IPMP (also known as 4-isopropyl 3-methylphenol, biosol or p-thymol) and mixtures thereof.

Oral healthcare compositions containing a source of zinc ions are also known for use in improving gum health and combating oral malodour.

JP2006176416 (Lion Corporation) describes an oral care composition comprising IPMP and a metal ion-carrying zeolite abrasive material. Such compositions exhibit high sterilization effects particularly on bacterial plaque found in the oral cavity.

U.S. Pat. No. 4,022,880 (Vinson et al) describes a composition for inhibiting dental plaque and calculus formation comprising a composition containing a source of zinc ions and a non-toxic organoleptically acceptable antibacterial agent. The use of IPMP is not described.

GB 1,373,003 (Unilever Ltd.) describes and claims a dentifrice composition having activity against plaque and calculus comprising a sparingly water-soluble zinc salt and a surfactant mixture of an alkali metal alkyl sulphate with either an alkali metal alkaryl sulphonate or an alkali metal alkyl ether sulphonate. Such compositions show reduced astringency.

U.S. Pat. No. 5,316,758 (Morishima et al) describes an oral care composition which exhibits dental plaque-inhibiting and gingivitis-preventing effects comprising a non-ionic antimicrobial agent (such as triclosan, thymol or IPMP) and certain amphoteric surface active agents. Such compositions have been shown to remain in the mouth over extended periods.

U.S. 2008/0253976 (Procter & Gamble) describes personal care compositions for oral, throat and skin care comprising a blend of a first component selected from citral, neral, geranial, geraniol and nerol and a second component selected from eucalyptol, eugenol and carvenol, which blend is described to exhibit both antibacterial and anti-inflammatory activities, stated to be particularly effective against bacteria-mediated inflammatory diseases such as gingivitis. Optionally the blend may further comprise additional antimicrobial and/or anti-inflammatory components including amongst many other potential agents, IPMP.

US 2007/0053849 (Procter & Gamble) describes topical oral care compositions comprising the combination of an anti-inflammatory agent with an antibacterial agent. Examples of anti-inflammatory agents include vitamin compounds; curcuminoids; oils and extracts from spices and botanicals; oils and extracts from thyme, oregano and sage; neem oil; flavonoids and flavones; and phenolics from plant sources. Examples of antibacterial agents include cetyl pyridinium chloride, stannous ion agent, zinc ion agent, copper ion agent, iron ion agent, triclosan, ascorbyl stearate, oleoyl sarcosine, dioctyl sulfosuccinate, alkyl sulphate and mixtures thereof. The use of IPMP is not described.

It has now been found that a composition comprising IPMP, a source of zinc ions, and an anionic surfactant has improved antibacterial activity when compared to compositions comprising as a single agent IPMP, a source of zinc ions or an anionic surfactant.

Without wishing to be bound by theory it is believed that the anionic surfactant increases the cell wall permeability of oral bacteria enabling IPMP and zinc ions to be taken up by such bacteria causing their death, or retarding their growth or metabolism.

In addition it has been found that a composition comprising IPMP has intrinsic anti-inflammatory activity, which activity is enhanced by the presence of a source of zinc ions.

Accordingly the present invention provides a composition comprising an antibacterial system comprising IPMP, a source of zinc ions and an anionic surfactant.

In one embodiment the composition of the present invention is a disinfecting composition.

In another embodiment the composition of the present invention is a pharmaceutical composition comprising a pharmaceutically acceptable carrier or excipient.

Suitable pharmaceutical dosage forms for oral administration include tablets and capsules. Suitable pharmaceutical dosage forms for topical administration include creams and ointments which can be applied to the skin.

Examples of pharmaceutically acceptable carriers or excipients are described in the Handbook of Pharmaceutical Excipients (eg the Fourth Edition, 2003, published by the Pharmaceutial Press).

In another embodiment the composition of the present invention is a personal care composition for oral, throat or skin care comprising a carrier or excipient acceptable for personal care use. Examples of suitable personal care dosage forms and carriers or excipients are described in U.S. 2008/0253976 (Procter & Gamble), the contents of which are herein incorporated by reference.

In a preferred embodiment the composition of the present invention is an oral care composition comprising an orally acceptable carrier or excipient.

Compositions of the present invention show particularly good bacterial kill with organisms most commonly found in the oral cavity, as shown in the data below.

Such oral care compositions are therefore of use in maintaining healthy gums and teeth and are of use combating oral health conditions caused or exacerbated by the presence of bacteria present in the oral cavity. In particular the oral care compositions of the present invention may help to keep the gum seal tight to teeth, thereby locking out plaque bacteria and protecting teeth above and below the gum surface, ie providing whole tooth protection.

In addition compositions of the invention will help prevent or remove surface deposited stains from natural teeth and dental prostheses.

A further advantageous property of the compositions of the invention includes combating halitosis (oral malodour or bad breath) that originates in the oral cavity.

Suitably the IPMP is present in an amount from 0.01% to 1.00%, for example from 0.04 to 0.20% or 0.05% to 0.10% by weight of the total composition.

Suitably the source of zinc ions, as defined as the zinc portion of a corresponding salt, is present in an amount from 0.01% to 2.50%, for example from 0.04% to 0.70% by weight of the total composition.

Suitably the source of zinc ions is a zinc salt such as zinc chloride, zinc citrate, zinc acetate, zinc sulphate, zinc gluconate, zinc salicylate, zinc lactate, zinc malate, zinc maleate, zinc tartrate, zinc carbonate, zinc phosphate, zinc oxide or zinc sulphate. Additional zinc salts are referred to in the above noted Vinson et al patent (U.S. Pat. No. 4,022,880).

A preferred zinc salt is zinc chloride.

Compositions of the present invention may comprise a buffering agent which can complex with the zinc ions thereby helping to reduce any untoward interactions with formulation excipients which could otherwise reduce the availability of the zinc ions. Examples of such buffering agents include citric acid/sodium citrate buffer. Suitably these are present in an amount to provide a pH of the composition of the present invention of less than pH 7.5 for example less than pH 6.5

Suitably the anionic surfactant is present in an amount from 0.1% to 15%, for example from 0.5% to 2.5% or for example 0.75% to 2.0% by weight of the total composition

Suitable examples of anionic surfactants include alkali metal C₈₋₁₈alkyl sulphates (eg sodium lauryl sulphate, SLS), alkali metal C₈₋₁₈alkylaryl sulphonates (eg sodium dodecylbenzene sulphonate, SDDBS), alkali metal sulphonated monoglycerides of C₁₀₋₁₈alkyl fatty acids (eg sodium coconut monoglyceride sulphonate), alkali metal C₁₀₋₁₈alkyl sulphoacetates (eg sodium lauryl sulphoacetate), and alkali metal salts of sarcosinates, isethionates and taurates, such as sodium lauryl sarcosinate, sodium lauroyl sarcosinate, sodium myristoyl sarcosinate, sodium palmitoyl sarcosinate, sodium stearoyl sarcosinate, sodium oleoyl sarcosniate and sodium lauroyl isethionate.

Suitably the anionic surfactant is an alkali metal C₈₋₁₈alkyl sulphate, an alkali metal C₈₋₁₈alkylaryl sulphonate or an alkali metal sarcosinate or a mixture thereof.

Most suitable anionic surfactants for use in the present invention are SDDBS, SLS, sodium lauryl sarcosinate and mixtures thereof, preferably in total concentration of 0.1% to 2.5%, more preferably 0.5% to 2.0%, even more preferably 1.0% to 1.5% by weight of the composition.

Suitably the pH of the composition is from pH 5.0 to 8.0, such as from 5.0 to 7.5, for example from 5.5 to 6.5.

In addition to the above ingredients, compositions of the present invention may comprise one or more active agents conventionally used in dentifrice compositions, for example, a fluoride source, a desensitising agent, an anti-plaque agent; an anti-calculus agent, a whitening agent, an oral malodour agent, an anti-inflammatory agent, an anti-oxidant, an anti-fungal agent, wound healing agent or a mixture of at least two thereof. Such agents may be included at levels to provide the desired therapeutic effect.

Suitable sources of fluoride ions for use in the compositions of the present invention include an alkali metal fluoride such as sodium fluoride, an alkali metal monofluorophosphate such a sodium monofluorophosphate, stannous fluoride, or an amine fluoride in an amount to provide from 25 to 3500 pm of fluoride ions, preferably from 100 to 1500 ppm. A typical fluoride source is sodium fluoride, for example the composition may contain 0.1 to 0.5% by weight of sodium fluoride, eg 0.204% by weight (equating to 927 ppm of fluoride ions), 0.2542% by weight (equating to 1150 ppm of fluoride ions) or 0.315% by weight (equating to 1426 ppm of fluoride ions).

Such fluoride ions help promote the remineralisation of teeth and can increase the acid resistance of dental hard tissues for combating caries, dental erosion (ie acid wear) and/or tooth wear.

In order to treat dental hypersensitivity, compositions of the present invention may comprise a desensitising agent. Examples of desensitising agents include a tubule blocking agent or a nerve desensitising agent and mixtures thereof, for example as described in WO02/15809 (Block). Examples of desensitising agents include a strontium salt such as strontium chloride, strontium acetate or strontium nitrate or a potassium salt such as potassium citrate, potassium chloride, potassium bicarbonate, potassium gluconate and especially potassium nitrate.

A desensitising agent such as a potassium salt is generally present between 2% to 8% by weight of the total composition, for example 5% by weight of potassium nitrate may be used.

Compositions of the present invention may comprise a whitening agent, for example selected from a polyphosphate, eg sodium tripolyphosphate (STP) and/or any additional silica abrasive present may have high cleaning properties. STP may be present in an amount from 2% to 15%, for example from 5% to 10% by weight of the total composition. Examples of high cleaning silica abrasives include those marketed as Zeodent 124, Tixosil 63, Sorbosil AC39, Sorbosil AC43 and Sorbosil AC35 and may be present in suitable amounts for example up to 20%, such as from 5 to 15% by weight of the total composition.

Compositions of the present invention will contain additional formulating agents such as abrasives, thickening agents, humectants, flavouring agents, sweetening agents, opacifying or colouring agents, preservatives and water, selected from those conventionally used in the oral hygiene composition art for such purposes.

To aid the foaming characteristics of the formulation, zwitterionic, amphoteric and non- or low-ionic surfactants may be used in addition to the anionic surfactant.

Examples of amphoteric surfactants include, long chain alkyl betaines, such as the product marketed under the tradename ‘Empigen BB’ by Albright & Wilson, long chain alkyl amidoalkyl betaines, such as cocamidopropylbetaine, alkyl ampho (di)acetates or low ionic surfactants such as sodium methyl cocoyl taurate, which is marketed under the trade name Adinol CT by Croda, or a mixture of at least two thereof.

Suitably, the additional surfactant or surfactants is/are present in the range 0.1% to 15%, for example from 0.5% to 10% or from 1.0% to 5% by weight of the total composition

Suitable humectants for use in compositions of the invention include glycerin, xylitol, sorbitol, propylene glycol or polyethylene glycol, or mixtures of at least two thereof; which humectant may be present in the range from 10% to 80%, for example from 20% to 70% or from 30% to 60% by weight of the total composition.

The compositions according to the present invention may be prepared by admixing the ingredients in the appropriate relative amounts in any order that is convenient and if necessary adjusting the pH to give a final desired value.

The pH is measured when the composition is slurried with water in a 1:3 weight ratio of the composition to water.

It will be understood that compositions of the present invention may also be used outside the oral cavity, for the cleaning of dentures and the like.

The oral composition of the present invention are typically formulated in the form of toothpastes, sprays, mouthwashes, gels, lozenges, chewing gums, tablets, pastilles, instant powders, oral strips, buccal patches, wound dressings, dental adhesives and the like.

When the composition is in the form of a toothpaste, it is suitable for containing in and dispensing from a laminate tube or a pump as conventionally used in the art. Additional examples may include bag-in-can or bag-on-valve delivery systems that utilise a foaming agent such as pentane or iso-pentane.

A typical process for making the composition of this invention involves admixing the ingredients, suitably under a vacuum, until a homogeneous mixture is obtained, and adjusting the pH if necessary.

The invention will now be described by way of the following non-limiting examples.

### EXAMPLE 1

### Antimicrobial Testing

### MIC Test

### Method

The MIC of a material composition was determined by the following method. A fresh culture of the test inoculum of each bacterium was diluted in sterile 0.1% special peptone solution to give a concentration of approximately 10⁶ colony forming units (cfu) per ml. Test samples of material were diluted in sterile tryptone soya broth (TSB) to give an initial stock solution, typically of 1% or 2% (10,000 or 20,000 ppm). However, it will be appreciated that the concentration of the initial stock solution of material can be varied if desired to investigate a different range of concentrations. Each row of a standard, 96-well plastic microtitre plate (labelled A-H) was allocated to one sample, i. e. eight samples per plate. Row H contained only TSB for use as a bacterial control to indicate the degree of turbidity resulting from bacterial growth in the absence of any test material.

Aseptically, 200 μl of the initial dilution of material was transferred to the 1^{st} and 7^{th} well of the appropriate row. All other test wells were filled with 100 μl of sterile TSB using an 8- channel micro-pipette. The contents of each of the wells in column 1 were mixed by sucking samples up and down the pipette tips, before 100 μl was transferred to column 2.

The same sterile pipette tips were used to transfer 100 μl of each well in column 7 into the appropriate well in column 8. This set of eight tips was then discarded into disinfectant solution. Using eight fresh, sterile tips the process was repeated by transferring 100 μl from column 2 into column 3 (and into 8 and 9). The process was continued until all the wells in columns 6 and 12 contained 200 μl. After mixing, 100 μl was discarded from wells in columns 6 and 12 to waste. Finally, 100 μl of pre-diluted bacterial test culture (approx 10⁶ cfu/ml) was added, thus giving a final volume of 200 μl in each well.

A blank plate was prepared for each set of eight samples in exactly the same way, except that 100 μl of sterile TSB was added instead of the bacterial culture. This plate was used as the control plate against which the test plate (s) could be read.

Test and control plates were then sealed using autoclave tape and incubated at 37° C. for 24 hours. The wells were examined after 24 hours for turbidity to determine if the material had inhibited growth or not. Plates are then read in a suitable microtitre plate reader at an absorbance of 540 nm as a measure of turbidity resulting from bacterial growth. The control, un-inoculated plate for each set of samples was read first, and the plate reader then programmed to use the control readings to blank all other plate readings for the inoculated plates for the same set of test materials (i. e. removing turbidity due to material and possible colour changes during incubation). Thus, the corrected readings generated were absorbances resulting from turbidity from bacterial growth.

### MIC Test Results

| | MIC (parts per million) | | |
|---|---|---|---|
| Organism | IPMP | SDDBS | Zn Gluconate |
| Streptococcus mutans | 1250 | 10 | 6250 |
| Staphylococcus aureus | 156 | 20 | 6250 |
| Escherichia coli | 312 | 625 | 1560 |

The MIC test results are presented above, and show that all of the agents tested have some inherent antimicrobial effects. These effects vary significantly between different bacterial strains, with both S. mutans and S. aureus highly sensitive to the surfactant SDDBS, but relatively tolerant of IPMP and Zinc. In contrast, E. coli is relatively insensitive to effects of SDDBS, but more susceptible to IPMP and Zinc.

### Kill Time Suspension Test

The method described herein allows the evaluation of in vitro antimicrobial efficacy by a kill time suspension test. A suspension of the test organism in the presence or absence of a solution of interfering substances is added to a sample of the product that has been diluted in hard water. The mixture is maintained at 20° C., or other temperatures appropriate to product use. After appropriate contact times an aliquot of the test mixture is taken. The antimicrobial activity of the aliquot is immediately neutralised by the dilution-neutralisation method. The number of surviving organisms from the test mixture and from the suspension of test organism is enumerated and the reduction in viable counts is calculated.

Materials 5% v/v Blood Agar (BA) (for Streptococcus mutans, Actinomyces viscosus and Fusobacterium nucleatum)

Tryptone Soy Agar (for Escherichia coli, Staphylococcus aureus)

Diluent—0.1% peptone,

Neutralisation medium—Letheen broth

### Hard Water (375 ppm as CaCO₃)

### Solution A

Dissolve 19.84 g of anhydrous MgCl₂ and 43.24 g of anhydrous CaCl₂ in purified water and make up to 1 litre using a volumetric flask.

### Solution B

Dissolve 35.02 g of NaHCO₃ in purified water and dilute to 1 L using a volumetric flask.

To 600 ml of purified water add 6 ml of solution A, and 8 ml of solution B. Dilute to 1 L using a volumetric flask. Sterilise the final solution by passing it through a membrane filter with an effective pore size of 0.45 μm. The final pH of the solution shall be 7.0±0.2 at 25° C. and should be adjusted where necessary using 0.5M HCl or 0.5M NaOH.

### Test Conditions

Dissolve 3 g of Bovine Serum Albumin (BSA) (Sigma, A-3425) in 100 ml of purified water. Sterilise by passing through a membrane filter with an effective pore size of 0.45 μm.

### Preparation of Test Cultures

From working cultures stored at 2-8° C. primary cultures of Streptococcus mutans, Escherichia coli, Actinomyces viscosus, Fusobacterium nucleatum and Staphylococcus aureus are grown on slopes of appropriate agar.

Transfer several loops of growth from the secondary culture to an appropriate diluent (0.1% peptone or other) and homogenize by vortex mixing. Adjust the concentration of the suspension prepared in so the optical density of the solution at 550 nm is equivalent to approximately 0.2.

A decimal serial dilution series of test suspensions are prepared (using 0.1% peptone) from 1:10 to 1:100,000. Duplicate plate counts are carried out by pour plating (S. aureus, E. coli) or spread plating (S. mutans, F. nucleatum, A. viscosus) 0.1 ml aliquots of the appropriate dilutions. Plates are incubated for appropriate periods (approximately 24 hours for S. aureus, E. coli; approximately 72 hours for S. mutans, F. nucleatum and A. viscosus). After incubation count each plate to calculate and record the mean cfu/ml of the original suspension.

Samples and toothpastes are tested at ¼ dilution (25% w/w). Initially, samples or toothpastes are prepared in hard water at a concentration of 1.25 times that required in the test. This allows for the dilution of the product that occurs during testing. Samples are prepared in sterile containers and volume sufficient to test each organism should be prepared (8 ml per organism).

The assessment of microbiocidal activity is carried out at room temperature (approximately 20+/−2° C., 1 ml of the test organism suspension is added to 1 ml of artificial saliva, and is then vortexed for 5 seconds. This is set aside for approximately 2 minutes. 8 ml of test product is added, a timing clock started and immediately vortexed for 5 seconds. After appropriate contact times (30 seconds or 120 seconds) a 1 ml aliquot is removed and added to 9 ml of neutralisation media to give a 1:10 dilution. This dilution is vortex mixed for 5 seconds and allowed to neutralize for at least 5 minutes. Further serial dilutions of 1 ml in 9 ml are made of the neutralised mixture, and 0.1 ml aliquots dispensed as appropriate into pour plates (E. coli, S. aureus), or spread plates (F. nucleatum, S. mutans, A. viscosus). After appropriate incubation, the number of bacteria on the plates is recorded, ideally at dilutions with 30-300 colonies per agar plate. All experiments should be replicated with independently prepared bacterial suspensions.

In order to validate the neutralization procedure, serial 1:10 dilutions of the test organisms are prepared to give concentration of approximately 10⁵ cfu/ml. To 8 ml of ‘Test Sample’ add 1 ml of sterile purified water and 1 ml of synthetic saliva. This is the ‘validation solution’. 1 ml of water is added to 9 ml of neutralisation medium (positive control), and 1 ml of ‘validation solution’ to a second 9 ml of neutralisation media (test). After approximately 5 minutes neutralisation time 0.1 ml of the diluted test organism suspension is added to each, and the mixtures vortexed and left for at least 5 minutes. The neutralised mixture is diluted 1:10 in diluent and duplicate plate counts performed of both the undiluted and 1:10 dilution, using appropriate agar and incubation conditions. After incubation count each plate and record the mean cfu/ml of the organism present. Neutralisation is considered valid if the control and test counts are within 0.3 Log10 cfu/ml of each other. If neutralisation is not valid dilution may be increased to 1 in 100.

The mean number of survivors is calculated for the each test and appropriate control samples, and expressed as the log to the base 10 (Log count). Where plates have no survivors the count is considered to have 0.5 colonies on that dilution for the purpose of calculation. The “log kill” is then calculated by subtracting the log survivors of the test solution from the log count of the untreated control solution. Data are presented below. Mean log kill is defined as the mean of log kill values determined in independent experiments.

Materials were tested both individually and in various combinations in the Kill Time assay. A range of microorganisms were used in these tests, including organisms typical of dental plaque (Streptococcus mutans, Fusobacterium nucleatum and Actinomyces viscosus) and standard reference organisms (Escherichia coli and Staphylococcus aureus) typical of faecal or skin bacteria, respectively.

Kill Time data at 30 s and 120 s for each organism in turn is shown in Graph 1 for Streptococcus mutans, Fusobacterium nucleatum and Actinomyces viscosus and for Escherichia coli and Staphylococcus aureus in Graph 2.

### Kill Time Data

Data are presented for three oral organisms: A .viscosus, F. nucleatum and S. mutans (Graph 1) and for two standard organisms E. coli, S. aureus (Graph 2). The following solutions were tested:

IPMP ¼ dilution of 0.1% w/w in 10% ethanol

SDDBS ¼ dilution of 1% w/v aq

Zinc Gluconate ¼ dilution of 1.25% w/v aq.

Results

For A. viscosus the results for both IPMP and Zinc alone show a kill of <0.5 log in all cases. SDDBS showed a significant kill of >3 log units at both 30 s and 120 s. Combination of IPMP/Zn/SDDBS produced >4 log units kill at both 30 s and 120 s (Graph 1).

For F. nucleatum IPMP alone showed limited effects. Both Zinc (around 1 log kill) and SDDBS (up to >3 log kill) showed significant effects. The combination of the three agents also produced maximum kill, with the higher IPMP level combined with SDDBS/Zinc producing maximum kill even at the shorter 30 s time point (Graph 1).

For S. mutans both IPMP and Zinc produced non-significant kill (<0.5 log units). SDDBS produced very high kill levels, with the 120 s time point showing maximum >5 log kill. The triple combination of IPMP(0.1%)/Zn/SDDBS showing the best effect (>4.5 log kill) (Graph 1).

For E. coli none of the three agents individually produced high levels of kill (kill of <0.3 log units in all cases). The triple combination, in contrast, showed synergistic effects, particularly with the higher level of IPMP combined with SDDBS/Zinc which showed kill of 1.3 log units at 30 s and almost 2 log units at 120 s (Graph 2).

For S. aureus both IPMP alone (at 0.1%) and SDDBS alone produced significant kills (>2 log). Zinc was ineffective alone. The triple combination gave the best results, with >4 log kill in all cases, and maximum kill (>5 log) at both 30 s and 120 s time points with the higher level of IPMP (Graph 2).

Kill Times for Toothpastes

The killing effect of a combination of IPMP/Zinc chloride/SDDBS/SLS (total of 1.0% surfactant) compared with standard SLS (1.5% surfactant) toothpaste is presented in Graph 3. The data presented above show that the benefit of triple combinations of IPMP and zinc salt together with surfactant is also detectable in dentifrice compositions.

Comparison of SLS/IPMP/ Zinc citrate versus SLS/IPMP and a standard SLS toothpaste is presented in Graph 4. The data presented above show that the benefit of triple combinations of IPMP and zinc salt together with surfactant is also detectable in whole dentifrices.

### Conclusion

The above data show the significant beneficial effect of combining surfactants such as SDDBS, SLS or both, with Zinc and IPMP to deliver better antibacterial effects in distinct antibacterial growth inhibition tests (MIC) or kill time assays, both in simple solutions and in dentifrice formulations.

### EXAMPLES 2 to 5

| Dentifrice Composition | Ex 2 | Ex 3 | Ex 4 | Ex 5 |
|---|---|---|---|---|
| Raw Material | % w/w | % w/w | % w/w | % w/w |
| Sorbitol, Liquid (Non-Crystallising) | 27.65 | 27.65 | 27.65 | 27.65 |
| Glycerin (98%) | 4.00 | 4.00 | 4.00 | 4.00 |
| Polyethylene Glycol 300 (PEG 6) | 4.00 | 4.00 | 4.00 | 4.00 |
| Silica, Dental Type (Zeodent 113) | 14.00 | 14.00 | 14.00 | 14.00 |
| Silica, Dental Type (Zeofree 153B) | 9.00 | 9.00 | 9.00 | 9.00 |
| Sodium Lauryl Sulphate | 0.75 | 0.75 | 1.50 | 1.50 |
| Sodium dodecylbenzenesulphonic | 0.75 | 0.75 | — | — |
| acid | | | | |
| Xanthan Gum (“xanth”, Keltrol F) | 0.80 | 0.80 | 0.80 | 0.80 |
| Carrageenan (“carra”, Genuvisco | 0.40 | 0.40 | 0.40 | 0.40 |
| TPH-1) | | | | |
| Saccharin Sodium | 0.30 | 0.30 | 0.30 | 0.30 |
| Sodium Fluoride | 0.24 | 0.243 | 0.20 | 0.10 |
| Zinc Chloride | 0.50 | 0.50 | 0.50 | 0.50 |
| Titanium Dioxide | 1.00 | 1.00 | 1.00 | 1.00 |
| Flavour | 1.50 | 1.50 | 1.50 | 1.50 |
| Sodium citrate tribasic dihydrate | 1.84 | 1.84 | — | 1.84 |
| Isopropylmethyl phenol | 0.05 | 0.10 | 0.05 | 0.1 |
| Citric acid (anhydrous) | — | — | 0.03 | 0.03 |
| Purified Water | ad | ad | ad | ad |
| | 100 | 100 | 100 | 100 |

### EXAMPLES 6-9

| Mouthwash Composition | Ex 6 | Ex 7 | Ex 8 | Ex 9 |
|---|---|---|---|---|
| Raw Material | % w/w | % w/w | % w/w | % w/w |
| Sorbitol, Liquid (Non-Crystallising) | 10.00 | 15.00 | 10.00 | — |
| Glycerin (98%) | 10.00 | 15.00 | 10.00 | 10.00 |
| Polyethylene Glycol 60 hydrogenated | 1.50 | 1.50 | 1.50 | 1.00 |
| castor oil | | | | |
| Sodium dodecylbenzenesulphonic acid | 1.00 | 1.50 | 1.00 | 0.50 |
| Saccharin Sodium | 0.03 | 0.03 | 0.03 | 0.03 |
| Sodium Fluoride | 0.06 | 0.06 | 0.06 | 0.06 |
| Zinc Chloride | 0.05 | 0.15 | 0.10 | 0.10 |
| Flavour | 0.20 | 0.20 | 0.20 | 0.25 |
| Sodium citrate tribasic dihydrate | 1.00 | 0.70 | 0.60 | 0.50 |
| Isopropylmethyl phenol | 0.10 | 0.05 | 0.01 | 0.01 |
| Methylparaben | 0.15 | 0.10 | 0.15 | 0.15 |
| Propylparaben | 0.15 | 0.10 | 0.15 | 0.15 |
| Bisabolol | 0.05 | 0.01 | 0.05 | 0.05 |
| Purified Water | ad | ad | ad | ad |
| | 100 | 100 | 100 | 100 |

1. A composition comprising an antibacterial system comprising 4-isopropyl-3-methyl phenol (IPMP), a source of zinc ions and an anionic surfactant.

2. A composition according to claim 1 which is an oral care composition comprising an orally acceptable carrier or excipient.

3. A composition according to claim 1 wherein the anionic surfactant is an alkali metal C₈₋₁₈alkyl sulphate or an alkali metal C₈₋₁₈alkylaryl sulphonate or an alkali metal sarcosinate or a mixture thereof.

4. A composition according to claim 3 wherein the anionic surface active agent is either SDDBS, SLS or sodium lauryl sarcosinate or a mixture thereof.

5. A composition according to claim 1 wherein the source of zinc ions is selected from zinc chloride, zinc citrate, zinc acetate, zinc sulphate, zinc gluconate, zinc salicylate, zinc lactate, zinc malate, zinc maleate, zinc tartrate, zinc carbonate, zinc phosphate, zinc oxide or zinc sulphate.

6. A composition according to claim 1 wherein the IPMP is at levels from 0.01% to 1.0% by weight of the total composition.

7. A composition according to claim 1 wherein anionic surfactant is at levels from 0.1% to 15% by weight of the total composition.

8. A composition according to claim 1 wherein the source of zinc ions, as defined as the zinc portion of a corresponding salt, is present in an amount from 0.01% to 2.5% by weight of the total composition.

9. A composition according to claim 1 comprising a source of fluoride ions.

10. A compositions according to claim 8 wherein the fluoride ion source is sodium fluoride.

11. A composition according to claim 1 comprising a desensitising agent.

12. A composition according to claim 1 comprising a whitening agent.

13. A composition according to claim 1 comprising an oral malodour agent.

14. A composition according to claim 1 in the form of a toothpaste.

15. A composition according to claim 1 in the form of a mouthwash.
